(19) United States
(12) Patent Application Publication
(10) Pub. No.: US 2021/0257070 A1
(43) Pub. Date: Aug. 19, 2021

(54) SYSTEM AND METHOD FOR PREVENTING WRONG-SITE SURGERIES

(71) Applicant: STARTBOX, LLC, Atlanta, GA (US)

(72) Inventors: Kaveh KHAJAVI, Brookhaven, GA (US); David E. LANE, II, Falkville, AL (US); John KERWOOD, Canton, GA (US); Austin BRITT, Hunstville, AL (US); Scott BLAND, Lexington, SC (US); Timothy DENTRY, McKinney, TX (US); Christopher DAVIS, Arlington, VA (US); Luke PERKINS, Spartanburg, SC (US); Dustin BACK, Houston, TX (US); Jonathan SPANGLER, San Diego, CA (US)

(21) Appl. No.: 17/251,881

(22) PCT Filed: Jun. 12, 2019
(86) PCT No.: PCT/US2019/036821

§ 371 Date: Dec. 14, 2020

Related U.S. Application Data
(60) Provisional application No. 62/684,150, filed on Jun. 12, 2018.

(51) Int. Cl. G16H 10/65; G16H 20/40

(52) U.S. Cl. CPC G16H 10/65; G16H 20/40

(57) ABSTRACT

A system and related methods of preventing wrong-site surgeries and sharps-related injuries to OR personnel, which includes a computer software program (for use on computers or hand-held devices in the medical environment) in combination with a surgical supply carrier. The surgical supply carrier may be associated with a patients medical chart (e.g. integrally or otherwise attached to) and is configured to lockably receive therein one or more surgical supplies (e.g. instruments, implants, and/or essential components) needed for the patients imminent medical procedure. The surgical supply carrier may further comprise an unlocking feature requiring at least two “keys” to unlock the carrier, with each “key” operating to verify that the correct surgical supplies are associated with the correct patient.

### CROSS-REFERENCES TO RELATED APPLICATIONS

The present application is an international application claiming the benefit of priority from commonly owned and co-pending U.S. Provisional Application Ser. No. 62/684,150 filed on Jun. 12, 2018 and entitled “UNIVERSAL LATERALITY LABELING SYSTEM AND RELATED METHODS,” the entire contents of which is hereby incorporated by reference into this disclosure as if set forth fully herein.

### BACKGROUND

A persistent safety issue is that of needle-stick and other sharps-related injuries to OR personnel, including scalpel or blade-related injuries. The Center for Disease Control estimates that each year approximately 385,000 needle-stick and other sharps-related injuries (averaging over 1000 a day), of which blade-related injuries account for almost 10%. Scalpel blades are necessarily extremely sharp and, as a result, are more likely to penetrate the flesh of a surgeon or other OR personnel more deeply than needle-stick injuries. Blade-related injuries can therefore be monumental for OR personnel, including contracting diseases stemming from blood-borne pathogens such as HIV/AIDS, hepatitis-C, hepatitis-B, etc., as well as the loss of income during recovery and rehabilitation and the potential loss of occupation due to permanent physical injuries (e.g. to the hand of a surgeon).

Wrong-site surgeries are also a persistent problem within the healthcare system. As defined by the Joint Commission on Accreditation of Healthcare Organizations (JCAHO), wrong-site surgery includes wrong side or site of the body, wrong procedure, and wrong-patient surgeries. A multitude of factors have been identified that may contribute to an increased risk of wrong-site surgery. Despite the implementation of strategies to prevent wrong patient, wrong site, wrong side surgery, regrettably this seemingly most preventable of complications still occurs. The incorrect assumption of a medical professional's infallibility, coupled with organized medicine's focus on the individual's medical mistakes rather than a systems approach have contributed to this problem.

In an attempt to improve patient safety, compliance with the Universal Protocol for Preventing Wrong Site, Wrong Procedure, Wrong Person Surgery is required of all Joint Commission accredited organizations. As a part of the universal protocol, a “pause” or “time out” is required. This serves as a final verification of: (1) the correct patient; (2) the correct procedure, site and side; and as applicable, (3) the availability of implants or instrumentation, prior to making incision. This is a time when all members of the surgical team are supposed to pause to review the case, and agree that the correct procedure is being done on the correct patient, at the correct site, and on the correct side. In theory, this would ensure that any errors that had been made could be detected prior to incision. In reality, the “time out” does not always occur; and when it does, not in any uniform or regular manner. Without a uniform or regular procedure, ritualized compliance, i.e. going through the motions, results in many institutions. The universal protocol cannot enforce a pause, and does not specify a protocol as to what should happen during a pause. The universal protocol does not specify a particular time for the pause to occur, and it does not specify a protocol as to what should happen during the pause; that is to say, what information should be communicated by whom, and to whom. While guidelines may be suggested, each institution determines how to comply, and therefore standardization is not achieved.

The present disclosure is directed at addressing the unmet needs of preventing or reducing wrong-site medical treatments.

### SUMMARY

The present disclosure addresses the unmet needs described above by providing a universal labeling system that provides quick and verifiable confirmation of laterality, as well as instantly accessible patient data. The present disclosure further describes a surgical supply carrier that may be associated with a patient's medical chart (e.g. integrally or otherwise attached to) and configured to lockably receive therein one or more surgical supplies (e.g. instruments, implants, needles, and/or essential components) needed for the patient's imminent medical procedure. The universal labeling system is described herein by way of example only in the context of a safety-blade dispenser suitable for use independently from or in conjunction with a system and method for preventing wrong-site surgeries such as that shown and described in commonly owned and co-pending International Patent Application PCT/US16/55210 (filed 3 Oct. 2016) entitled “System and Method for Preventing Wrong-Site Surgeries”, published as WO2017-059452 on Apr. 6, 2017 (“the '210 PCT”), the contents of which is hereby incorporated by reference as is set forth herein in its entirety. However, it should be understood that the universal labeling system described herein is universally applicable to any sterile object (e.g. blades, needles, implants, and the like) used during any type of medical, surgical (e.g. orthopedic, ophthalmic, and the like), and/or dental procedure.

By way of example only, the surgical supply carrier is suitable for use independently from or in conjunction with the system and method described in the '210 PCT. The surgical supply carrier may further comprise an unlocking feature requiring at least two “keys” to unlock the carrier, with each “key” operating to verify that the correct surgical supplies are associated with the correct patient.

By way of example, the '210 PCT describes a system and method of preventing wrong-site surgeries by utilizing a computer software program (e.g. set of instructions) executed by a computer-implemented electronic device (e.g. comprising a screen for displaying images, an optical scanner for receiving data, a processor operable to execute instructions, and a data storage medium for storing instructions) to perform certain tasks that enable a user or group of users to track and verify accuracy of information and medical equipment pertaining to a particular patient's imminent medical procedure. The computer software program can be run on any of a variety of computing devices, such as a computer (e.g. stationary desktop and/or laptop) and/or a hand-held computing device (e.g. smart-phones such as IPHONE and/or a tablet device such as an IPAD or SURFACE PRO) used within the medical environment. The “medical environment” includes anywhere along the continuum in which patient and medical team (including the doctor, office personnel, nurses, medical technicians, surgeons, administrators) interact, from the doctor's office (where the initial consultation and decision for treatment is made) to the operating room (where the surgery takes place, if necessary). The term may also include personnel involved with post-treatment data collection and/or analysis, such as (but not limited to) (a) insurance companies for the patient, hospital and/or doctor, (b) state and/or federal agency departments/programs (e.g. Medicare/Medicaid) which reimburse funds to the hospital and/or doctor, (c) any other agency (private and/or governmental) which generates payment to the patient, hospital and/or doctor for the specific case, and/or (d) quality control and/or hospital administration to identify areas of improvement and/or best practices.

The computer software program supports and provides several functionalities, including but not necessarily limited to voice recording, video recording, recording playback, an electronic patient-identifying component (such as a patient ID band) capable of being scanned, a sterile medical article (e.g. safety-blade dispenser, surgical supply carrier) capable of being scanned (for example using a QR code), and any of a variety of analytics generated or based upon data acquired through the use of the system from “decision-to-incision,” that is, from the decision to pursue a particular treatment protocol (made in the doctor's office) through the actual treatment (e.g. surgery in the OR). Moreover, the computer software program may be configured to use artificial intelligence and/or machine learning to generate predictive analytics, prescriptive analytics, and/or data-based best practices. Scanning of the patient ID band and/or sterile medical article may be accomplished using the scanning functionality of the computer, hand-held device, wearable device, and/or scanning systems separate from the system that cooperate and communicate with the system. The system may use any of a variety of suitable biometric identification technologies (e.g. iris scan, finger-prints, genetics, etc.) in order to identify the patient (and/or the guardian of the patient if the patient is a minor or incapacitated) at any point in the medical environment.

The software program may be utilized by multiple users and at multiple stages of the overall process, including but not limited to the initial decision in a doctor's office to have surgery, scheduling, hospital admissions, pre-op, anesthesia, OR preparation, and most significantly, a final “time-out” immediately prior to first incision. For example, the decision for surgery typically occurs in the surgeon's office after one or more consults and assessments of the patient's pathology and surgical options. The system allows the surgeon to create a patient profile for that particular patient within the software program located on the surgeon's computer (e.g. desktop, laptop, or mobile device), which may include any of a variety of information regarding the intended surgery. This intended-surgery information may include (but is not necessarily limited to) patient name, date of birth, procedure type, procedure location, procedure laterality, surgeon name, and a voice (and/or video) recording created by the surgeon (through the use of the software program) as he or she dictates aloud the intended-surgery information. The voice (and/or video) recording may also include the patient's consent to the intended surgery. The software program also saves the time and date that the patient profile was created, as well as the individual in the surgeon's office who created it.

When an open date and time is selected, the surgery scheduler uses the software program (e.g. via computer) to update the patient profile to include the date, time and location of the scheduled surgery, which may then be communicated to the surgeon's office and the patient to ensure its on their respective calendars. Importantly, the surgery scheduler may utilize the audio/video playback of the system to watch/listen to the original recording of the surgeon as he or she dictated the intended-surgery information at the decision stage. This recording playback feature provides an advantageous cross-check on the scheduling process, in that it allows the surgery scheduler to replay the original recording to ensure: (a) the information communicated by the surgeon's office (e.g. by phone) matches that of the original recording; and/or (b) the information received from the surgeon's office (e.g. by phone) was accurately entered into the software program by the surgery scheduler. The software program also saves the time and date that the patient profile was updated, as well as the individual in surgery scheduling who updated it. Once the surgery has been scheduled, the software program can be configured to send out a link to the patient via email, text SMS, or another electronic mechanism, which contains a written notification with details (along with date, time, location of surgery and any pre-admission restrictions, such as no-food, time to arrive at admissions, parking instructions) of the surgery or link directing the patient to an online patient portal to retrieve the content. The software program may be configured to send certain information pertaining to the surgery to other key personnel (e.g. third party device representatives, monitoring personnel, cell saver, etc.). The system may also be used to track any pre-surgical clearances and/or assessments, such as (but not limited to) cardiac and/or blood work-ups that may need to be conducted and successfully passed before the day of surgery.

During the hospital admissions process, after showing appropriate identification (e.g. driver's license, passport, etc. . . . ) to admissions personnel and/or using patient-identification biometrics, the patient is assigned an identifying device, such as patient identification (ID) band capable of being attached or coupled to the patient in some manner, most commonly around the wrist of the patient. The patient ID band includes a bar code with identifying information such as patient name, date of birth, and social security number. The admissions personnel may then update the patient profile within the software program to include the patient information from the patient ID band, such as by using a scanner in electronic communication with the software program. The admissions personnel may also use the audio/video playback feature of the software program (e.g. via computer) to listen to the original recording of the surgeon to double check that the intended-surgery information in the software program (as entered by the surgeon's office and updated by the surgery scheduler) is accurate and consistent with the intended-surgery information of the original recording. This represents yet another cross-check on the integrity of the information in the software program and ensure the scheduled surgery is as intended. The software program also saves the time and date that the patient profile was updated, as well as the individual in admissions who updated it.

The next stage involves the patient checking in to the pre-operative stage or department (so-called “pre-op”) within the location of the scheduled surgery. When the patient arrives at pre-op, pre-op personnel will use the software program (e.g. via hand-held device) to distribute and assign an appropriate safety blade-dispenser to the patient. To do so, pre-op personnel will use a scanner to scan the patient ID band, which will then bring up the patient profile for that particular patient. With the patient profile revealed, pre-op personnel may then select and distribute an appropriate safety blade-dispenser depending upon the laterality of the intended surgery (e.g. rose-colored for a right-sided surgery, lavender-colored for a left-sided surgery, or neutral-colored (such as grey) for a procedure without laterality). Pre-op personnel will then use the scanner to scan a unique identifier on a label disposed on the safety blade-dispenser, which may take the form of a unique serial number stored in a data-storage feature on the label, such as (but not necessarily limited to) a barcode or QR code. The software program may then be used by pre-op personnel to link the data from the patient ID band to the data on the label of the safety blade-dispenser. In this fashion, the specific safety blade-dispenser is assigned to the specific patient, which facilitates accurate tracking of the safety blade-dispenser the rest of the way through the medical environment. The surgeon may use the audio/video recording functionality to amend the patient profile to include a second recording associated with any add-on surgery he or she decides to do during the pre-op assessment. The second recording may also include the patient consent to the add-on surgery.

Pre-op personnel may also use the audio/video playback feature to watch/listen to the original recording of the surgeon to double check that the intended-surgery information saved in or by the software program (as entered by the surgeon's office and updated by the surgery scheduler, admissions personnel and optionally any pre-surgery work-up assessment/clearance) is accurate and consistent with the intended-surgery information of the original recording. This represents yet another cross-check on the integrity of the information saved in or by the software program and ensure the scheduled surgery is as intended. If a problem is detected, pre-op personnel may select “No Go” functionality within the software program. The surgeon may review and override if he or she decides the problem has been resolved or, alternatively, cancel the surgery if he or she decides the problem has not been resolved or surgery should not go forward as scheduled. The software program also saves the time and date that the patient profile was updated, as well as the individual in pre-op who updated it.

Anesthesia personnel may then use the system to confirm they are to administer anesthesia to the correct patient for the correct or intended surgery. This is an optional step and merely illustrates how the system and methods of preventing wrong-site surgeries and blade-related injuries may be used by any of a variety of hospital or OR-personnel throughout the medical environment. Anesthesia personnel may use the audio/video playback feature of the system to listen to the original recording, scan the patient ID band and/or scan the label of the assigned safety blade-dispenser to ensure the patient and the intended-surgery are consistent with the original recording of the surgeon, a voice recording for any add-on surgery decided upon and consented during pre-op and/or with the anesthesia plan. If a problem is detected, the anesthesia personnel may select “No Go” functionality within the software program. The surgeon may review and override if he or she decides the problem has been resolved or, alternatively, cancel the surgery if he or she decides the problem has not been resolved or surgery (original and/or add-on) should not go forward as scheduled. The software program also saves the time and date that the patient profile was updated, as well as the individual in anesthesia who updated it.

The next stage involves OR preparation (so-called “OR prep”) wherein the patient is transferred from pre-op to the OR and prepared for surgery. When the patient is transferred from pre-op to the OR, OR personnel will use the software program (e.g. via hand-held device) to scan the patient ID band and/or the label of the safety blade-dispenser that was assigned to the patient in pre-op. OR personnel will then inspect the data resulting from this scanning and compare it to, among other things, the laterality color-coding of the safety blade-dispenser to ensure the correct patient has the correct and assigned safety blade-dispenser. OR personnel may also use the audio/video playback feature of the software program to wacth/listen to the original recording of the surgeon to double check that the intended-surgery information in the software program (as entered by the surgeon's office and updated by the surgery scheduler, any optional pre-surgical work-up/clearance, admissions personnel, pre-op personnel, and the surgeon during pre-op) is accurate and consistent with the intended-surgery information of the original recording (from the surgeon's office) and any second recording (from pre-op). This represents yet another cross-check on the integrity of the information in the software program and ensure the scheduled surgery is as intended. If a problem is detected, OR personnel may select “No Go” functionality within the software program. The surgeon may review and override if he or she decides the problem has been resolved or, alternatively, cancel the surgery if he or she decides the problem has not been resolved or surgery (original and/or add-on) should not go forward as scheduled. If the surgery is to go forward, the patient is then transferred to an OR table where they are prepared and draped for surgery. The software program saves the time and date that the patient profile was updated, as well as the OR personnel who updated it.

The final stage involves the time-out procedure that must be performed before the surgeon can access a blade from the safety blade-dispenser. A user (e.g. member of the medical team present in the OR such as an OR technician) first scans the label of the safety blade-dispenser to pull up the patient profile for the patient on the OR table. The user may review that data, and augment or double check that against the laterality color-coding of the assigned safety blade-dispenser and the audio/video replay of the original recording from the surgeon's office (which is played in a way that everyone in the OR can watch/hear and confirm the initial recorded decision) and any additional recording from pre-op.

If everything is correct and the surgeon decides to move forward with the surgery, he or she next performs the time-out by: (a) gaining the attention of everyone in the OR; and (b) stating the required time-out information such as (but not necessarily limited to) patient name, date of birth, procedure, laterality (if any), and surgeon name. The user preferably uses the audio/video recording feature of the software program to record as the surgeon speaks while performing the time-out, which becomes part of the patient profile. Once the time-out has been completed (and optionally recorded), the user may then use the software to indicate that the time-out has been performed (e.g. by toggling a switch and/or clicking a designated box within the software program or related app).

At that point, the surgeon (or another member of the medical team present in the OR) will remove the label from the safety blade-dispenser and place the label in or with the patient chart. By removing the label, a plurality of blades will now be accessible that were previously covered by the label. A blade count is made to document the number of blades held in the safety blade-dispenser, which blade information may be saved in the patient profile via the software program and/or in the patient chart. With the label removed a medical team member will be able to dispense one of a plurality of blades from the housing of the safety blade-dispenser, which can couple to a handle to allow the surgeon to make the initial incision. The software program saves the time and date that the patient profile was updated, as well as the OR personnel or surgeon who updated it.

The system and method of the '210 PCT includes a computer software program configured to provide one or more users with a method of preventing wrong site surgeries, in combination with any of the various safety-blade dispensers described therein. According to the present disclosure, the system and method of the '210 PCT may be used in conjunction with a surgical supply carrier and/or any sterile object used during any type of medical procedure (e.g. invasive, non-invasive, in-patient, and/or out-patient), for example including but not limited to surgical blades, catheter kits, implants, needles, ocular lens replacement, specimens, and the like, whether provided by the manufacturer/distributor of the computer software program or by a third-party medical device supplier. The sterile object can optionally include at least one component, such as a label, paper, tape, or packaging, which prevents or impedes a surgeon from accessing one or more sterile medical articles stored within until after a “time-out” is performed by the physician or authorized medical personnel to confirm various details (e.g. correct patient, correct procedure, correct equipment, etc.) before starting the intended medical procedure.

The sterile medical object can include any sterile medical object used in a desired patient treatment protocol. The sterile medical object (and/or label described below) can optionally be color-coded to indicate the laterality of the surgery (e.g. rose or red for “right” sided surgery, lavender for “left” sided surgery, and a neutral color (such as grey) for a surgery with no-laterality). The sterile medical object (and/or its packaging) can optionally include a label with a QR code capable of being scanned and linked with patient-data from the patient ID band via the software assembly to create a unique identifier for the particular sterile medial object assigned to the patient during the pre-treatment assessment in the hospital after admission on the day of treatment. This unique identifier ensures that the patient receives the correct type of sterile medical object, meaning the correct laterality of the intended treatment, and can be tracked throughout the remainder of the medical environment and associated with any data captured throughout the entire medical environment to ensure it is correct and used to perform the intended treatment. The label can only be removed from the sterile medical object after a “time out” has been performed by the doctor or authorized medical personnel. Once the label is removed, the doctor then and only then has access to the sterile medical object, and the treatment may be commenced.

The sterile medical object can optionally be initially provided sealed in transparent double sterile packaging (which is then placed in a non-sterile container with a transparent window). The transparent packaging/window allows for the identifying information on the confirmation label (e.g. QR code and/or laterality indicator) to be scanned before the sterile medical object is removed from any of the packaging. In this manner, one can avoid the need to have the same identifying information on multiple levels of the packaging. This reduces manufacturing costs and the complexity of matching multiple packaging components to ensure they all have the same identifying information, which would otherwise be required.

The universal laterality labeling system and system of preventing wrong-site surgeries allow for tracking of a variety of data from pre-hospitalization to the actual treatment procedure, which the software program can use to generate any of a variety of analytics. The analytics may be based upon, but not necessarily limited to, so-called “near miss” data (that is, errors that were caught and avoided during the use of the system), treatment type and laterality, treatment outcomes, treatment complications, patient demographics, geographic information, as well as the date, time, location and personnel associated with each interaction or use of the system for efficiency and accountability. For example, analytics based on “near miss” data may provide the hospital and/or insurers and/or quality improvement specialists valuable data as to where errors or possible errors may have occurred in order to drive remediation efforts to minimize or avoid such errors in the future. The analytics may also be used to identify best practices based on the data collected, either within the hospital system (“intra-system”) and/or amongst multiple different hospital systems (“inter-system”), and assessed to identify best practices for further reducing wrong-site surgery errors. Moreover, the computer software program may be configured to use artificial intelligence and/or machine learning to generate predictive analytics, prescriptive analytics, and/or data-based best practices.

The surgical supply carrier (or “lock box”) may be non-sterile and configured for placement within a treatment room (e.g. operating room), and further configured to hold therein essential instrumentation or other medical objects needed to perform the desired procedure, including but not limited to sterile tactical tools (in sterile packaging) as described above. By way of example, the surgical supply carrier may include a base having an internal compartment and a lid hingedly attached to the base at one side. The internal compartment is sized and configured to hold one or more medical objects that are specifically selected for a particular patient in a given procedure. The lid is sized and configured to block access to the internal compartment when the lid is in a closed position. The lid is moveable from a closed position in which access to the internal compartment is blocked to an open position in which access to the internal compartment is unencumbered. The surgical supply carrier further includes a locking mechanism configured to releasably lock the lid to the base when the lid is in the closed position. By way of example, each lockable compartment is opened with a unique key (e.g. a physical key or electronic code), that is provided, accessible, and/or green-lighted by the software program after the final timeout for each procedure has been recorded (e.g. in the cloud).

The non-sterile surgical supply carrier may be prepared during the pre-op stage. For example, when the patient arrives at pre-op, pre-op personnel will use the software program (e.g. via hand-held device) to distribute and assign at least one appropriate essential medical object (e.g. blade dispenser, implant, or other surgical item) to the patient. To do so, pre-op personnel will use a scanner (e.g. optical scanning functionality of hand-held device) to scan the patient ID band, after which the computer will cause the patient profile for that particular patient to be displayed on the display screen of the hand-held device). With the patient profile revealed, pre-op personnel may then select and distribute (e.g. by placing in the internal compartment) one or more appropriate medical objects depending upon the particular requirements of the intended surgery. Pre-op personnel will then use the scanner to scan a unique identifier on a label disposed on the lock box, which may take the form of a unique serial number stored in a data-storage feature on the label, such as (but not necessarily limited to) a barcode or QR code. The software program (including cloud server) may then be used by pre-op personnel to link the data from the patient ID band to the data on the label of the lock box. In this fashion, the specific lock box, including the contents placed therein, is assigned to the specific patient, which facilitates accurate tracking of the lock box the rest of the way through the medical environment. As an additional level of tracking and verification, the pre-op personnel may also scan unique identifiers on labels disposed on each of the medical objects prior to placement within the lock box. In that manner, the computer (via software) may track and record information regarding the specific medical objects used during the procedure.

Once all of the intended medical objects have been scanned by pre-op personnel so that the computer may associate the medical objects with the patient profile, the pre-op personnel places the medical objects in the interior compartment of the lock box, and closes the lid thereby engaging the locking mechanism. At this point, the lock box is successfully locked and may not be opened until the final timeout has been recorded in the software (and/or the cloud). In one example embodiment, the lock box may be configured to require more than one “key” to enable unlocking. For example, the first “key” may be the requirement of a certain patient action confirming association of the lock box with the correct patient (as well as simultaneously confirming patient consent to the procedure). Such patient action may include (but not be limited to) a digitally-captured signature or biometric identification (e.g. iris scan, fingerprint, etc.) transmitted to and recorded by the cloud server. In another embodiment, the lock box comprises a portion of the patient's physical chart (e.g. the medical chart may be incorporated into or otherwise attached to the lid).

The lock box remains locked until its presence in the treatment room has been verified during the “time out” sequence just prior to treatment. After verification of the lock box by the computer (including cloud server), the lock box may be opened so that the contents may be retrieved from the internal compartment. This may be accomplished in several ways depending upon the technological capabilities of the specific lock box being used. For example, according to one embodiment, the lock box may be manually opened by a user after verification using a physical key that is inserted into a key hole on the lock box. According to another embodiment, the lock box may be equipped with a numerical keypad with which a user may input a code supplied by the computer after a successful lock box verification check to unlock the box. According to another embodiment, the lock box may be equipped with a digital control panel comprising among other things a microprocessor, user interface, and communications module having the ability to communicate with another computer, for example a handheld computer housing the software and/or cloud server that stores all relevant data pertaining to the procedure. Upon verification of the correct lock box, the computer may cause the handheld device or cloud server to communicate with the lock box, sending the unlock instructions directly to the lock box, which then unlocks automatically.

The lock box may be provided with one or more color indicators to convey certain information that can be recognized and verified on sight, for example such as a color indicator on the lock box that indicates laterality. In addition, a color indicator may be provided that displays a color to match a color-coded patient ID wristband, which may help reduce patient mixups. Another color indicator may be provided to indicate locked and unlocked status of the lock box.

As additional description to the embodiments described below, the present disclosure describes the following embodiments.

Embodiment 1 is a system for preventing or tracking a wrong-site error during a surgical procedure associated with an individual interacting within a medical environment, comprising: (1) a surgical supply carrier comprising at least one internal compartment configured to hold one or more surgical instruments or components to be used during said surgical procedure for said individual, a movable barrier preventing access to said internal compartment when said movable barrier is in a closed position, a locking mechanism configured to maintain the moveable barrier in said closed position until completion of at least two unlocking events, storage media comprising at least one of an electronic profile for said individual and electronic instructions for unlocking said surgical supply carrier, and a communications module configured to enable communication between said storage media and a processor; and (2) a computer implemented electronic device having a screen for displaying images, an optical scanner for receiving data, a processor operable to execute instructions, and a data storage medium for storing instructions which when executed by the processor cause the processor to: (a) save and display said electronic profile for said individual; (b) associate one or more surgical devices with said electronic profile; (c) receive and store a first data input comprising a first of said at least two unlocking events; (d) enable an electronically recorded time out to be conducted prior to starting of said surgical procedure associated with said individual, said time out comprising a second of said at least two unlocking events; and (e) access said storage media of said surgical supply carrier to enable execution of said electronic instructions for unlocking said surgical supply carrier.

Embodiment 2 is the system of embodiment 1, wherein the surgical supply carrier comprises a medical chart of said individual.

Embodiment 3 is the system of embodiment 2, wherein said medical chart is physically attached to said surgical supply carrier.

Embodiment 4 is the system of embodiment 2, wherein said medical chart is electronically stored within said storage media.

Embodiment 5 is the system of any of embodiments 1 through 4, wherein said surgical supply carrier further comprises a display component configured to display data contained within said storage media.

Embodiment 6 is the system of any of embodiments 1 through 5, wherein the optical scanner is configured to retrieve data from at least one of a QR code and a bar code.

Embodiment 7 is the system of any of embodiments 1 through 6, wherein the moveable barrier is hingedly attached to the surgical supply carrier.

Embodiment 8 is the system of any of embodiments 1 through 7, wherein said surgical supply carrier further includes at least one color indicator corresponding to at least one of laterality, patient identification, and lock status.

Embodiment 9 is the system of any of embodiments 1 through 8, wherein execution of said instructions for unlocking said surgical supply carrier causes said moveable barrier to be unlocked electronically.

Embodiment 10 is the system of any of embodiments 1 through 9, wherein execution of said instructions for unlocking said surgical supply carrier causes said computer implemented electronic device to display instructions to a user to manually unlock said moveable barrier.

Embodiment 11 is the system of any of embodiments 1 through 10, wherein said one or more surgical devices to be associated with said electronic profile each include at least one of a bar code and a QR code readable by said optical scanner.

Embodiment 12 is the system of any of embodiments 1 through 11, wherein said first data input comprises digital consent to continue with the surgical procedure, given by one of said individual and a legal guardian of said individual.

Embodiment 13 is the system of embodiment 12, wherein said surgical supply carrier further comprises a digital signature capture element in electronic communication with said storage media, said digital signature capture element configured to capture said digital consent.

Embodiment 14 is the system of any of embodiments 1 through 13, wherein said second of said at least two unlocking events comprises: (1) using said optical scanner to scan one of a bar code and a QR code affixed to said surgical supply carrier to retrieve data contained within said code; (2) communicating said retrieved data to said processor; and (3) comparing said retrieved data to said electronic profile to verify a match between said retrieved data and said electronic profile.

Embodiment 15 is the system of any of embodiments 1 through 14, wherein said electronic profile of said individual comprises at least one of data that identifies said individual, data that identifies said medical procedure scheduled to be performed on said individual, and an indication of procedural laterality.

Embodiment 16 is a method for preventing or tracking a wrong-site error during a surgical procedure associated with an individual interacting within a medical environment, comprising the steps of: (1) providing a surgical supply carrier, the surgical supply carrier comprising at least one internal compartment configured to hold one or more surgical instruments or components to be used during said surgical procedure for said individual, a movable barrier preventing access to said internal compartment when said movable barrier is in a closed position, a locking mechanism configured to maintain the moveable barrier in said closed position until completion of at least two unlocking events, storage media comprising at least one of an electronic profile for said individual and electronic instructions for unlocking said surgical supply carrier, and a communications module configured to enable communication between said storage media and a processor; (2) providing a computer implemented electronic device having a screen for displaying images, an optical scanner for receiving data, a processor operable to execute instructions, and a data storage medium for storing instructions which when executed by the processor cause the processor to save and display said electronic profile for said individual, associate one or more surgical devices with said electronic profile, receive and store a first data input comprising a first of said at least two unlocking events, enable an electronically recorded time out to be conducted prior to starting of said surgical procedure associated with said individual, said time out comprising a second of said at least two unlocking events, and access said storage media of said surgical supply carrier to enable execution of said electronic instructions for unlocking said surgical supply carrier; (3) associating said surgical supply carrier with said individual; (4) populating said internal compartment with one or more surgical components; (5) locking said moveable barrier in a closed position; (6) capturing said first data input comprising said first of said at least two unlocking events before the individual has been prepped for surgery; (7) performing said time out including performing said second of said at least two unlocking events while the individual is awaiting commencement of surgery; (8) retrieving said at least one surgical component from said internal compartment; and (9) performing said surgical procedure as scheduled on said individual.

Embodiment 17 is the method of embodiment 16, wherein said surgical supply carrier comprises a medical chart of said individual.

Embodiment 18 is the method of embodiment 17, wherein said medical chart is physically attached to said surgical supply carrier.

Embodiment 19 is the method of embodiment 17, wherein said medical chart is electronically stored within said storage media.

Embodiment 20 is the method of any of embodiments 16 through 19, wherein said surgical supply carrier further comprises a display component configured to display data contained within said storage media.

Embodiment 21 is the method of any of embodiments 16 through 20, wherein the optical scanner is configured to retrieve data from at least one of a QR code and a bar code.

Embodiment 22 is the method of any of embodiments 16 through 21, wherein the step of associating said surgical supply carrier with said individual comprises affixing an identification label including one of a bar code and a QR code to said surgical supply carrier, said one of a bar code and a QR code including at least one of data that identifies said individual, data that identifies said medical procedure scheduled to be performed on said individual, and an indication of procedural laterality.

Embodiment 23 is the method of any of embodiments 16 through 22, wherein said step of populating said internal compartment with one or more surgical components comprises the sub-steps of: (a) using said optical scanner to scan one of a bar code and a QR code associated with each surgical component to retrieve identifying data pertaining thereto; (b) communicating said retrieved identifying data to said processor; (c) associating said retrieved identifying data with said electronic profile of said individual; and (d) placing said surgical component into said internal compartment.

Embodiment 24 is the method of any of embodiments 16 through 23, wherein the moveable barrier is hingedly attached to the surgical supply carrier.

Embodiment 25 is the method of any of embodiments 16 through 24, wherein said surgical supply carrier further includes at least one color indicator corresponding to at least one of laterality, patient identification, and lock status.

Embodiment 26 is the method of any of embodiments 16 through 25, wherein execution of said instructions for unlocking said surgical supply carrier causes said moveable barrier to be unlocked electronically.

Embodiment 27 is the method of any of embodiments 16 through 26, wherein execution of said instructions for unlocking said surgical supply carrier causes said computer implemented electronic device to display instructions to a user to manually unlock said moveable barrier.

Embodiment 28 is the method of any of embodiments 16 through 27, wherein capturing said first data input comprises obtaining digital consent to continue with the surgical procedure, given by one of said individual and a legal guardian of said individual.

Embodiment 29 is the method of embodiment 28, wherein said surgical supply carrier further comprises a digital signature capture element in electronic communication with said storage media, said digital signature capture element configured to capture said digital consent.

Embodiment 30 is the method of any of embodiments 16 through 29, wherein said performing of said second of said at least two unlocking events comprises: (1) using said optical scanner to scan one of a bar code and a QR code affixed to said surgical supply carrier to retrieve data contained within said code; (2) communicating said retrieved data to said processor; and (3) comparing said retrieved data to said electronic profile to verify a match between said retrieved data and said electronic profile.

Embodiment 31 is the method of any of embodiments 16 through 30, wherein said electronic profile of said individual comprises at least one of data that identifies said individual, data that identifies said medical procedure scheduled to be performed on said individual, and an indication of procedural laterality.

### BRIEF DESCRIPTION OF THE DRAWINGS

Many advantages of the present disclosure will be apparent to those skilled in the art with a reading of this specification in conjunction with the attached drawings, wherein like reference numerals are applied to like elements and wherein:

FIG. 1 is a graphical representation of a wrong-site treatment prevention system including a universal laterality labeling system according to one embodiment of the disclosure;

FIG. 2 is a top plan view of an example of a sterile medical object forming part of the wrong-site treatment prevention system of FIG. 1 with a confirmation label forming part of the universal laterality labeling system of FIG. 1 attached thereto;

FIG. 3 is a top plan view of the sterile medical object with confirmation label of FIG. 2 shown sealed in transparent double sterile packaging;

FIG. 4 is a perspective view of the sterile medical object with confirmation label sealed in double sterile packaging of FIG. 3 shown sealed in non-sterile outer packaging;

FIG. 5 is a top plan view of another example of a sterile medical object forming part of the wrong-site treatment prevention system of FIG. 1 with another example of a confirmation label forming part of the universal laterality labeling system of FIG. 1 attached thereto;

FIGS. 6-8 are exemplary graphic user interface (GUI) screens of a software program forming part of the wrong-site treatment prevention system of FIG. 1, showing the process by which sterile medical objects are scanned into the software program, according to one embodiment of the disclosure;

FIG. 9 is a perspective view of a lock box forming part of the wrong-site treatment prevention system of FIG. 1;

FIG. 10 is a perspective view of a chart-associated lock box forming part of the wrong-site treatment prevention system of FIG. 1;

FIG. 11 is a plan view of one end of the chart-associated lock box of FIG. 10;

FIG. 12 is a graphical representation of a multiple unlock “key” capture and transmission protocol forming part of the wrong-site treatment prevention system of FIG. 1; and

FIGS. 13-14 are block diagrams of a computer systems forming part of the wrong-site treatment prevention system of FIG. 1.

### DETAILED DESCRIPTION OF A PREFERRED EMBODIMENT

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The system and method for preventing wrong-site surgeries disclosed herein boasts a variety of inventive features and components that warrant patent protection, both individually and in combination.

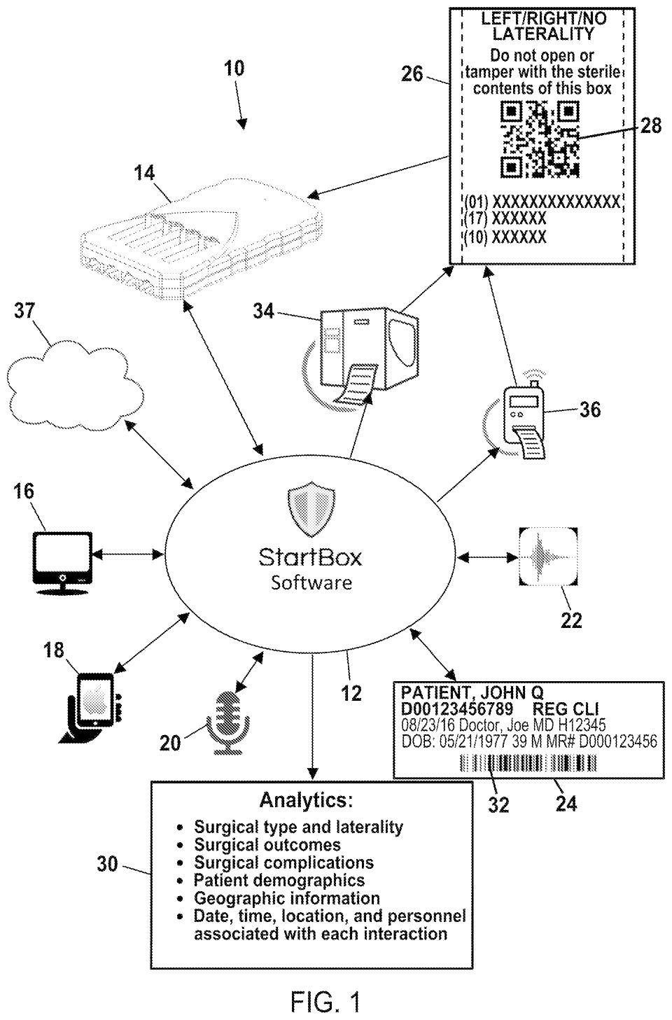

FIG. 1 is a graphical representation of an exemplary wrong-site treatment prevention system which may include any sterile object used during treatment, for example a safety-blade dispenser as shown and described in the '210 PCT for preventing or reducing sharps-related injuries to OR personnel. As shown in the example embodiment of FIG. 1, a system 10 includes computer software program 12 comprising a set of instructions that, when executed by a processor, provide a user with a method of preventing wrong site surgeries, in combination with a sterile medical object 14 used during treatment. The sterile medical object 14 used during treatment is shown and described herein by way of example only as a safety-blade dispenser 14 as shown and described in the '210 PCT (incorporated by reference), however any sterile medical object or “tactical tool” (e.g. blade, needle, implant, etc.) used during treatment may be used with the wrong-site treatment prevention system of the present disclosure. The sterile medical object used during treatment (e.g. safety-blade dispenser) 14 comprises at least one component, such as a label, paper, or tape, which prevents or impedes a doctor from using the sterile medical object (e.g. accessing one or more surgical instruments stored within) until after a “time-out” is performed by the doctor or authorized medical personnel before starting the intended medical procedure. As will be described below, the sterile object may alternatively be locked inside a non-sterile surgical supply carrier positioned within the treatment room (e.g., operating room, patient room, clinic room, and the like), and is accessible only after the final “time-out” is performed by the doctor or authorized medical personnel before starting the intended medical procedure.

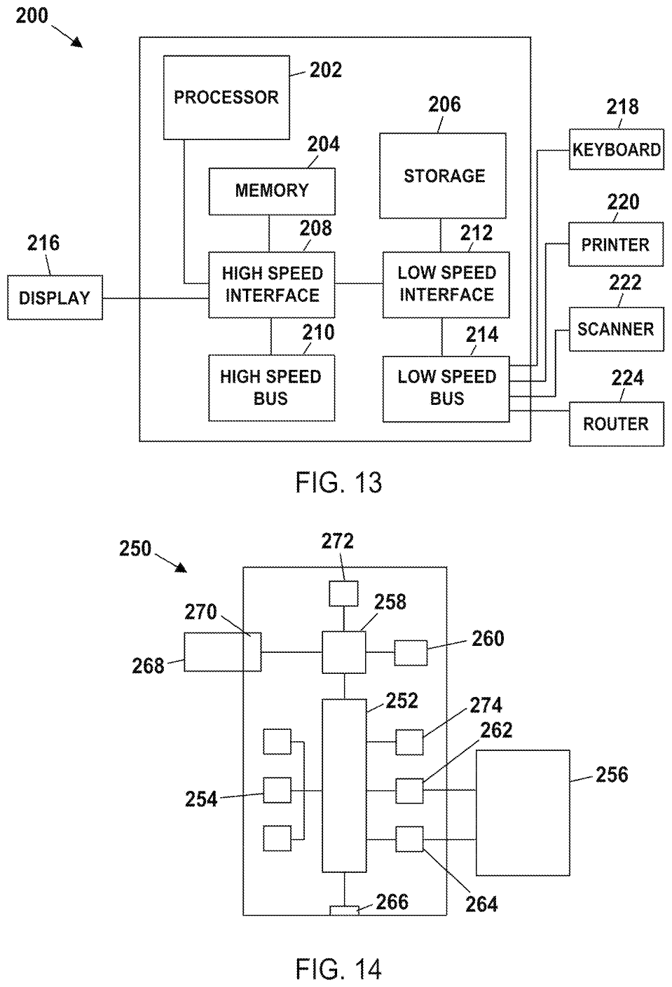

The computer software program 12 can be run on any of a variety of computer-implemented electronic devices (which may be collectively referred to herein using the generic term “computer”), such as a stationary desktop and/or laptop computer 16, a hand-held computing device 18 (e.g. smart-phones such as IPHONE and/or a tablet device such as an IPAD or SURFACE PRO), and/or a wearable device (e.g. a smart-watch such as APPLE WATCH) used within the medical environment. While computing devices are known in the art, such devices generally include a processor coupled to memory, and networking hardware. Computing devices may be operatively connected with the processor such that the processor can process network traffic inbound from the Internet and deliver outbound network traffic to the Internet utilizing, for example, a multi-layered networking protocol, such as TCP/IP. The processor is preferably connected to or may have input devices, such as a keyboard, mouse, or a touch screen display displaying alphanumeric and/or numeric symbols. A display unit, such as an LCD screen, may be used to display any data output. The memory may include both volatile and non-volatile memory, and stores program code executable by the processor. The program code causes the processor to perform various steps that direct each computer 16 and/or hand-held device 18 to perform one or more embodiment methods for preventing wrong site surgery. The program code may reside in permanent memory, such as on a hard disk, and then be loaded into non-volatile memory for execution, or may (for example) be obtained from a remote server via the networking hardware and then loaded into non-volatile memory for execution. Use of a computer database (not shown) for storing user-specific data and/or a program database may also be envisioned, although persons of ordinary skill routinely make use of alternative strategies for storing data for use by a processor. The computer 16 and/or hand-held device 18 may contain one or more speakers, microphones, cameras, or scanners. To aid in tracking capability, the hand-held device 18 may contain one or more tracking technologies, such as GPS (Global Positioning System) transmitters or receivers, RFID (Radio Frequency Identification) transmitters or receivers and/or other wireless tracking technology. The hand-held device 18 includes a power source, which may be any of a variety of suitable battery types including but not limited to a rechargeable Lithium battery. A USB port may be provided on computer 16 and/or hand-held device 18 to aid in powering the device and/or for transferring data. See FIGS. 13-14 and accompanying disclosure for further discussion of example computer systems suitable for use with the wrong-site treatment prevention system of the present disclosure.

The “medical environment” includes anywhere along the continuum in which patient and medical team (including the doctor, office personnel, nurses, medical technicians, surgeons, administrators) interact, from the doctor's office (where the initial consultation and decision for surgery is made) to the operating room (where the surgery takes place, if necessary) or any other treatment location. The term may also include personnel involved with post-treatment data collection and/or analysis, such as (but not limited to) (a) insurance companies for the patient, hospital and/or doctor, (b) state and/or federal agency departments/programs (e.g. Medicare/Medicaid) which reimburse funds to the hospital and/or doctor, (c) any other agency (private and/or governmental) which generates payment to the patient, hospital and/or doctor for the specific case, and/or (d) quality control and/or hospital administration to identify areas of improvement and/or best practices.

The system and related methods of preventing wrong-site treatment utilizes computer software program 12 (comprising instructions that when executed by a processor) support and provide several functionalities. These include, but are not necessarily limited to, voice recording 20, recording playback 22, an electronic patient-identifying component (such as a patient ID band 24 including a bar code 34) capable of being scanned, sterile object used during surgery (e.g. safety-blade dispenser) 14 capable of being scanned (e.g. by way of attached confirmation label 26 including a QR code 28), and any of a variety of analytics 30 generated or based upon data acquired through the use of the system 10 from “decision-to-incision,” that is, from the decision to have treatment (made in the doctor's office) through the actual treatment (e.g. surgery in the OR). Moreover, the computer software program may be configured to use artificial intelligence and/or machine learning to generate predictive analytics, prescriptive analytics, and/or data-based best practices. Scanning of the patient ID band 24 (e.g. by scanning a bar code 32) and/or sterile object 14 (e.g. by scanning a QR code 28) may be accomplished by the scanning functionality (e.g. optical scanner) of the computer 16, hand-held device 18, wearable device, and/or scanning systems separate from the system 10 which cooperate and communicate with the system 10 by way of cloud server 37, (e.g. an offsite computer storage medium in communication with the processor via the Internet). The system 10 may use any of a variety of suitable biometric identification technologies (e.g. iris scan, finger-prints, genetics, etc.) in order to identify the patient (and/or the guardian of the patient if the patient is a minor or incapacitated) at any point in the medical environment.

The confirmation label 26 may be printed using a stationary label printer 34, for example at a distributor prior to delivery to the treatment site, or a handheld label printer 36, for example at a treatment site after delivery but prior to treatment. The confirmation label 26 may be printed on a sticker-type material with an adhesive backing for affixation to the outside of packaging. Alternatively, the confirmation label 26 may be printed on paper stock with tear-away adhesive strips, for example for affixation directly to a sterile medical object for removal after the final “time out” immediately prior to use. The confirmation label 26 may be situated in such a way so as to impede access to the sterile medical object (as described herein with regard to the safety-blade dispenser 14), or alternatively may be situated such that access to the sterile medical object is not impeded but the confirmation label 26 serves as a visual reminder to the medical staff to follow protocol to final timeout. An important feature of the confirmation label 26 is the inclusion of a Quick Response (QR) code 28 and/or a bar code 34 (e.g. UPC code), each of which is a machine-readable optical label that may be programmed to contain information pertinent to the treatment. For example, such pertinent information may include (but is not limited to) patient information, treatment information (e.g. type of treatment, site of treatment), and physician name. Additionally, information pertaining to the sterile medical object itself may be included, for example including but not limited to manufacturer, distributor, manufacturing batch number, and serial number (if applicable). As such, a physician or medical support team member may scan the QR code 28 (or bar code 34) with a reader, optical scanner, or camera, which transmits coded information to the processor, which in turn translates the coded information and presents the information to the user via a GUI or other display, enabling the user to view the information programmed therein. The user may then instruct the computer via the software and graphic user interface to associate the information with a particular patient, for example by selecting a “link” icon on the GUI which then instructs the computer to link the information to the patient's electronic profile.

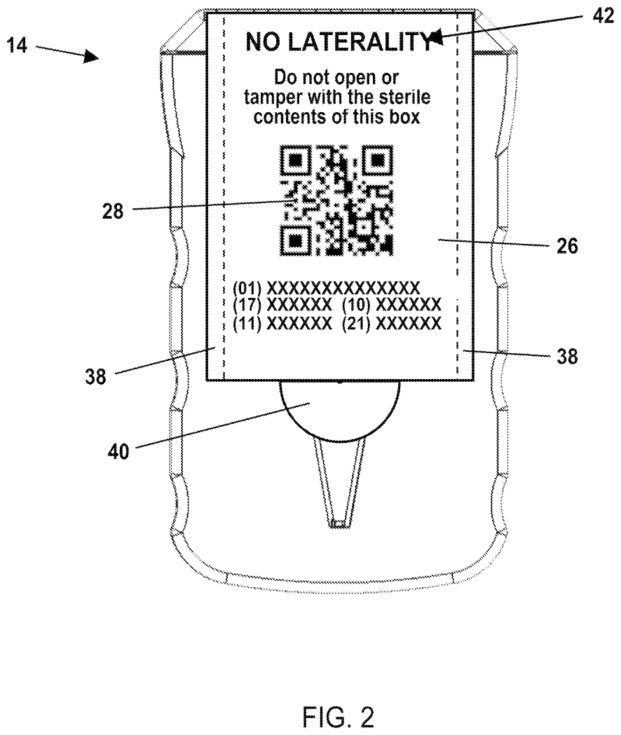

Referring to FIG. 2, an example of a sterile medical object in the form of a safety-blade dispenser 14 is provided with an example of a confirmation label 26 to help reduce the prevalence of wrong site surgeries. The confirmation label 26 is placed in a manner that renders the surgical blades contained within the dispenser 14 inaccessible unless and until the user removes the label 26. The confirmation label 26 does not have adhesive on it, but is affixed to the safety-blade dispenser 14 via attached sticky strips 38 from which the label 26 can be torn away. By way of example, the confirmation label 26 may include any suitable patient data printed on the label and/or contained in an electronically scannable code (e.g. Quick Response (QR) code 28, bar code 32, and the like) that the user must scan before removing the confirmation label 26. The confirmation label 26 further includes a pull-tab 40 to enable more efficient removal. In addition to patient data, the confirmation label 26 may include a laterality indicator 42 that immediately visually conveys to the user the laterality, if any, of the procedure. This laterality indicator 42 may include words and/or be color-coded. For example, the label may include the word “LEFT” and/or be colored lavender/purple to indicate a left side surgery, “RIGHT” and/or red color to indicate a right side surgery, and “NO LATERALITY” and/or gray color to indicate no laterality. Additionally, the outer edges of the confirmation label 26, for example including the adhesive strips 38 may be colored with the same color as the laterality indicator 42 to enhance visibility of the laterality-indicating color. Once the confirmation label 26 has been removed, it can be attached to the patient record by any suitable means.

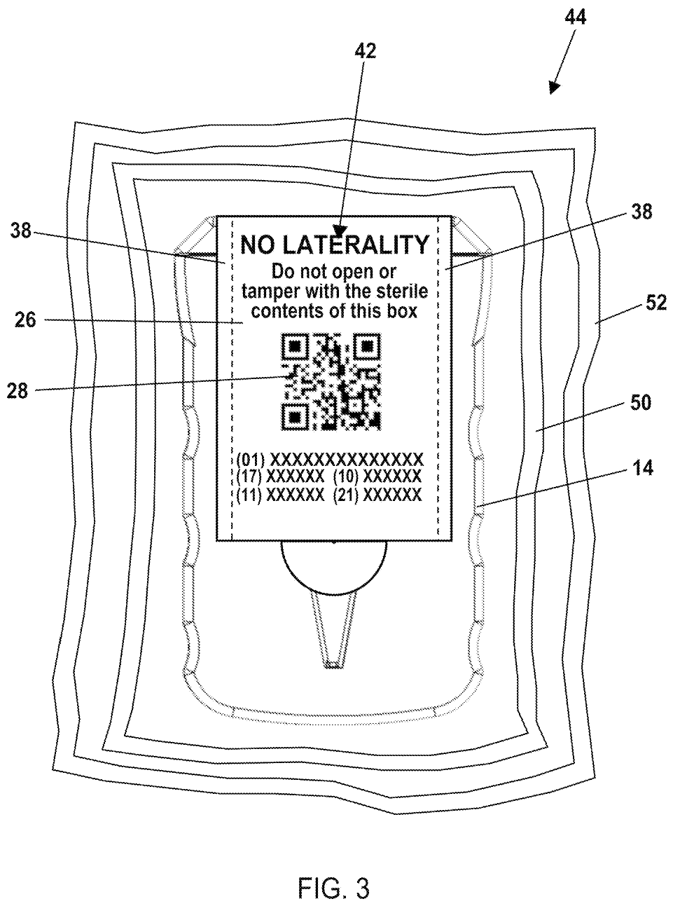

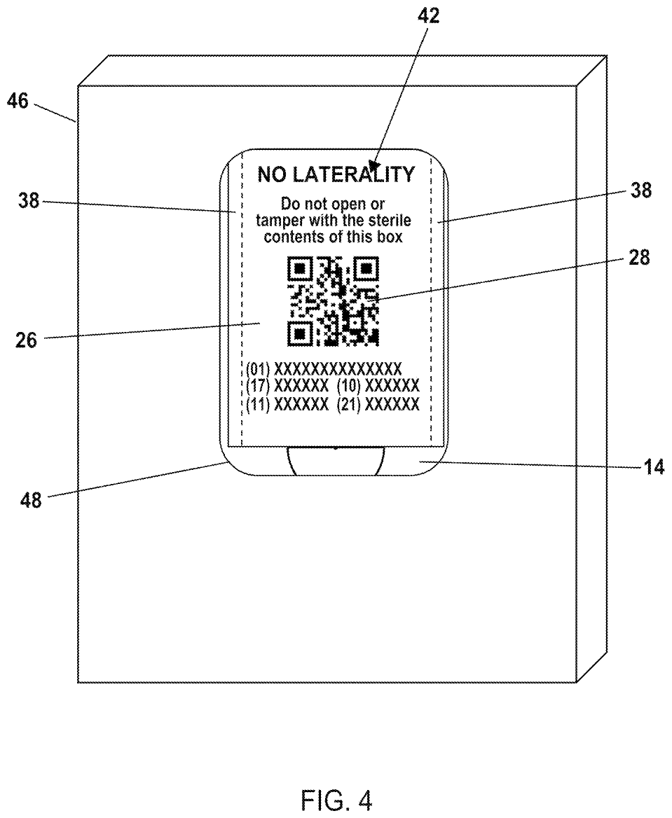

FIGS. 3-5 illustrate different examples of how the laterality-indicating confirmation label 26 may be associated with sterile medical objects and in particular the various packaging types. For example, as shown in FIGS. 3-4, the sterile medical object (e.g. safety-blade dispenser) 14 may be provided within double sterile packaging 44 disposed within a container 46 having a transparent window section 48. More specifically, the sterile medical object 14 is disposed within a first transparent sterile package 50, which is then sealed within a second transparent sterile package 52. The combined sterile packages 50, 52 are disposed within the container 46 such that identifying information on the confirmation label 26 (e.g. QR code 28 and/or laterality indicator 42) may be visible and scannable through the transparent window section 48 of the container 46 and the transparent first and second sterile packages 50, 52. In this manner, one can avoid the need to have the same identifying information reproduced on multiple layers of packaging (i.e. first sterile package 50, second sterile package 52, or outer container 46). This reduces manufacturing costs and the complexity of matching multiple packaging components to ensure they all have the same identifying information, which would otherwise be required but for the transparent sterile packages 50, 52 within the container 46 having the transparent window section 48 through which the QR code 28 (and/or bar code 34) on the label 26 may be scanned.

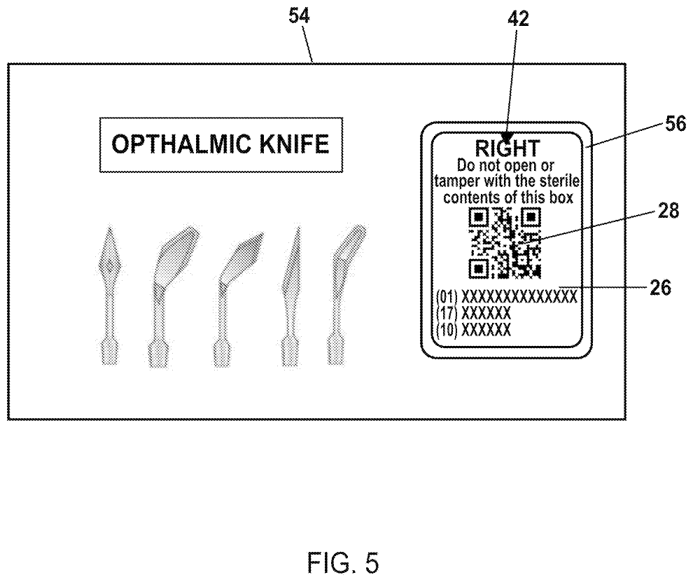

FIG. 5 illustrates an example of a laterality-indicating confirmation label 26 affixed to the outside of packaging 54 containing sterile medical objects 14. In the instant example, the sterile medical objects are ophthalmic knives, however the laterality-indicating confirmation label 26 of the present disclosure may be associated with any sterile medical object used during any treatment. The confirmation label 26 in this instance may be the sticker-type label 26 including a QR code 28, color-coded laterality indicator 42, and color-coded perimeter border 56. By way of example, the sticker-type label 26 may be used with third-party medical objects that may be identified and/or procured at the treatment site, as well as medical objects not also offered by the manufacturer/distributor of the wrong-site surgery prevention system.

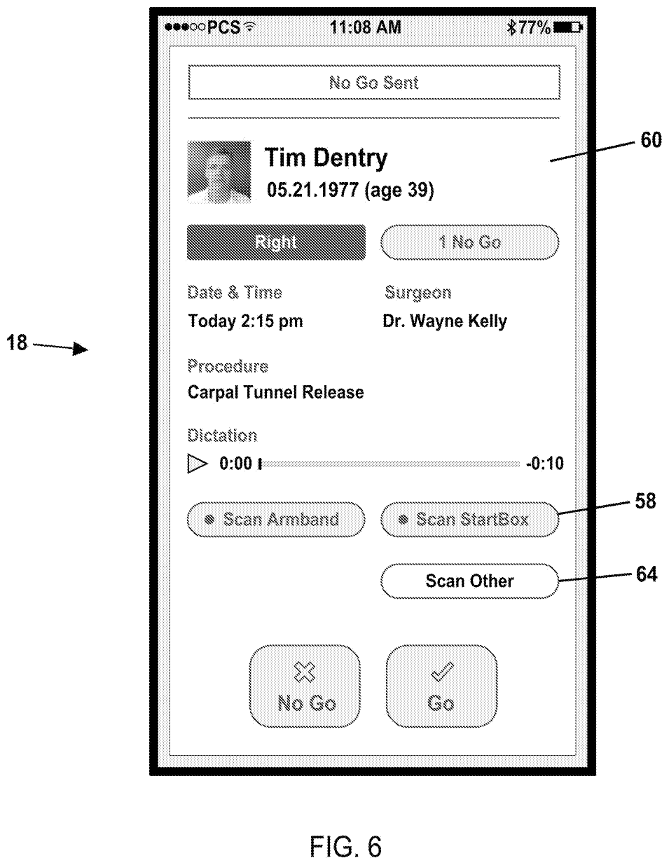

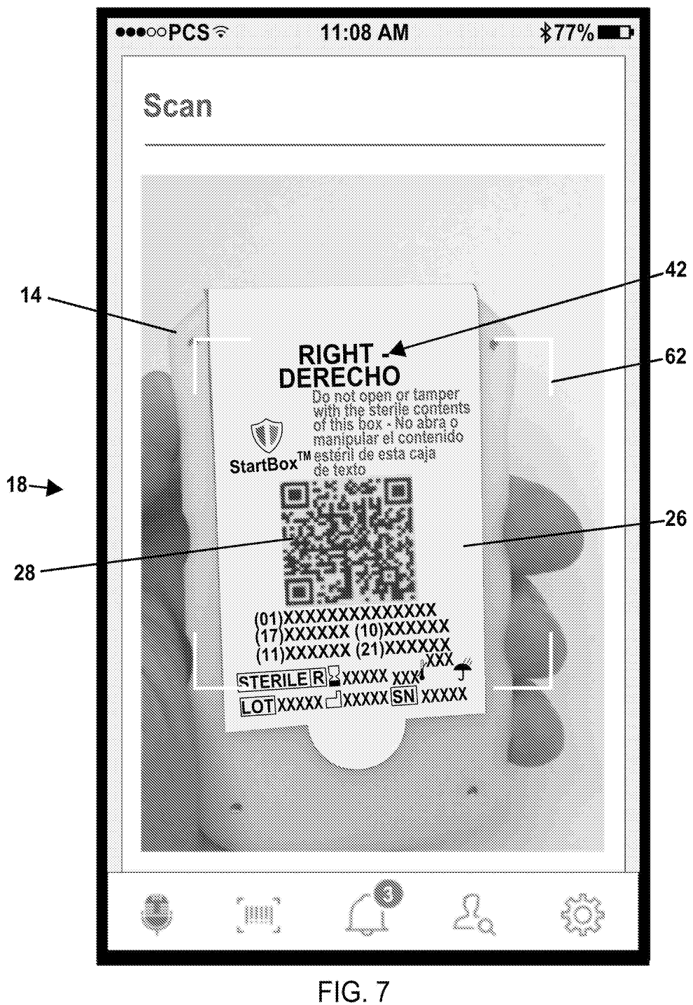

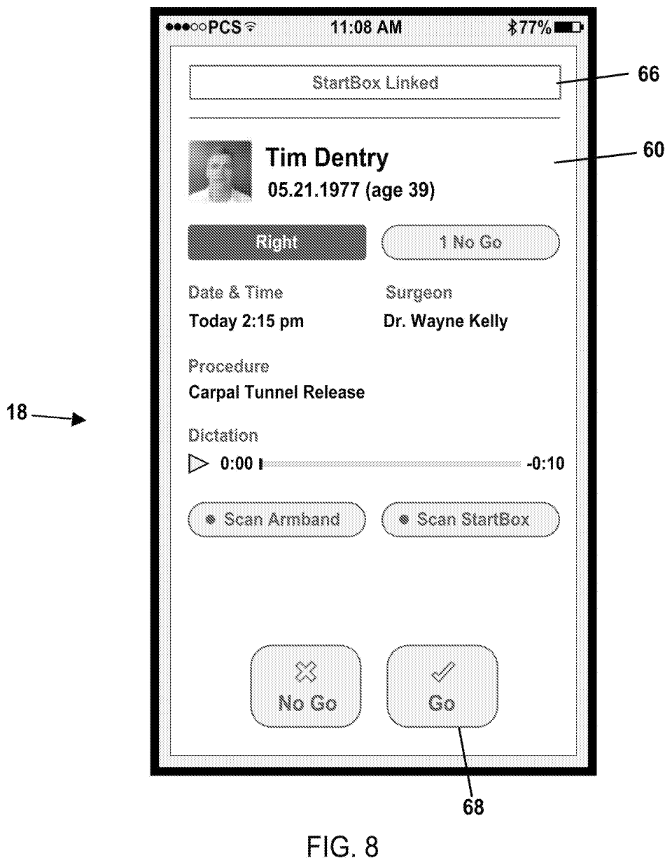

FIGS. 6-8 illustrate a series of exemplary graphic user interface (GUI) screens that a computer-implemented electronic device may present and a user may encounter while using the computer software program 12 on the electronic device (e.g. handheld device 18), in particular during the process of scanning a QR code 28 associated with a sterile medical object 14, including a third-party sterile medical object according to present disclosure. For example, to scan the sterile medical object 14, the user may select the “Scan StartBox” 58 icon on the example GUI screen 60 which instructs the computer to activate a scanner 62 (e.g. optical scanner) associated with or contained within the hand-held device 18. By way of example, the particular icon 58 that activates the scanner 62 may have words other than “Scan Startbox” (e.g. “Scan Medical Object”, “Scan Verification Code”, etc.), and/or the GUI screen 60 may include one or more icons (e.g. “Scan Other” icon 64) that that instruct the computer to activate the scanning feature of the handheld device 18 (or other platform running the software 12) but indicate use of a third-party sterile medical object (and/or surgical supply carrier 70 and/or chart-associated surgical supply carrier 90 described below). The computer may also record such usage information for tracking purposes (e.g. by transmitting the usage information to the cloud server 37 for storage where it may be accessible by any device in communication with the server). The QR code 28 of the sterile medical object 14, which has a laterality indicator 42 designating right-laterality via name (RIGHT) and red or rose color, may then be scanned by a scanner (e.g. optical scanner). The data contained therein is “read” by the computer and compared to the previously saved patient profile and/or intended-treatment information. If the laterality matches, computer links the sterile medical object 14 with the patient profile, and causes a “StartBox Linked” notification banner 66 to appear at the top of the GUI screen 60. In cases in which a third party sterile medical object 14 is used, then a banner indicating the name of the object and successful linking of that object may appear at the top of the GUI screen 60. The user may thereafter select the “GO” icon 68 to proceed to instruct the computer to advance to the next step.

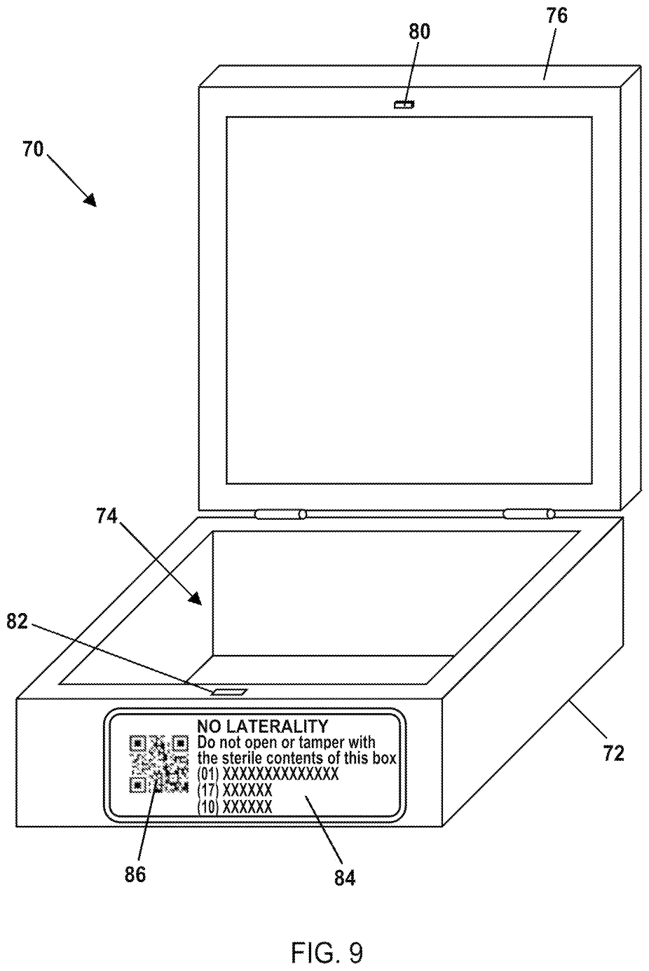

FIG. 9 illustrates one example of a non-sterile surgical supply carrier or “lock box” 70 configured for placement within a treatment room (e.g. operating room) and further configured to hold therein essential instrumentation or other medical supplies needed to perform the desired procedure, including but not limited to sterile tactical tools (in sterile packaging) as described above. By way of example, the lock box 70 shown and described herein includes a base 72 having an internal compartment 74 and a lid 76 hingedly attached to the base 72 at one side. The internal compartment 74 is sized and configured to hold one or more medical objects 14 that are specifically selected for a particular patient in a given procedure. The lid 76 is sized and configured to block access to the internal compartment 74 when the lid 76 is in a closed position. The lid 76 is moveable from a closed position in which access to the internal compartment 74 is blocked to an open position in which access to the internal compartment 74 is unencumbered. The lock box 70 further includes a locking mechanism 78 configured to releasably lock the lid 76 to the base 72 when the lid 76 is in the closed position. By way of example, the locking mechanism 78 may comprise a latch 80 on the lid 76 that engages a pin in a recess 82 on the base 72, however any appropriate locking mechanism may be used. Furthermore, although shown and described as a box with an interior compartment and a lid hingedly attached, the lock box 70 may comprise other types of lockable compartments in addition to or instead of the configuration shown herein, including but not limited to drawers, slideable lids, and the like. Additionally, the lock box 70 may have a plurality of lockable compartments containing essential instrumentation for multiple medical procedures, for use in situations where a single patient has several medical procedures (surgical or otherwise) scheduled for the same hospital visit. In this example, each lockable compartment is opened with a unique key (e.g. a physical key or electronic code), that is provided, made accessible, and/or green-lighted by the software program 14 (e.g. if all the information (e.g. patient data, tactical tool data, laterality information, etc.) is positively matched and verified, the cloud server 37 sends an unlock instruction/code 112 to the handheld device 18 that causes the computer to display instructions for the user to act on) after the final timeout for each procedure has been recorded (e.g. in the cloud 37).

By way of example only, the non-sterile lock box 70 may be prepared during the pre-op stage. For example, when the patient arrives at pre-op, pre-op personnel will use the software program 12 (e.g. via hand-held device 18) to distribute and assign at least one appropriate essential medical object 14 (e.g. blade dispenser, implant, or other surgical item) to the patient. To do so, pre-op personnel will use a scanner (e.g. optical scanning functionality of hand-held device 18) to scan the patient ID band 24, after which the computer will cause the patient profile for that particular patient to be displayed on the display screen of the hand-held device 18. With the patient profile revealed, pre-op personnel may select one or more appropriate medical objects 14 depending upon the particular requirements of the intended surgery. The one or more medical objects 14 is/are then associated with the patent profile, for example by manual data input by way of the graphic user interface. Alternatively, as an additional level of tracking and verification, the pre-op personnel may also scan unique identifiers (e.g. bar codes or QR codes) on labels disposed on each of the medical objects 14 (or packaging of said objects 14) prior to placement within the lock box 70. In that manner, the computer (via software 12) may track and record more detailed information regarding the specific medical objects used during the procedure. Pre-op personnel will then use the scanner to scan a unique identifier on a label 84 disposed on the lock box 70, which may take the form of a unique serial number stored in a data-storage feature 86 on the label, such as (but not necessarily limited to) a barcode or QR code. The software program 12 (including cloud server 37) may then be used by pre-op personnel to instruct the computer to link the data from the patient ID band 24 to the data on the label 84 of the lock box 70. In this fashion, the specific lock box 70, including the contents placed therein, is electronically assigned to the specific patient, which facilitates accurate tracking of the lock box 70 the rest of the way through the medical environment.

Once all of the intended medical objects 14 have been scanned by pre-op personnel so that the computer may associate the medical objects 14 with the patient profile, pre-op personnel places the medical objects 14 in the interior compartment 74 of the lock box 70, and closes the lid 76 thereby engaging the locking mechanism 78. At this point, the lock box 70 is successfully locked and may not be opened until the final timeout has been recorded in the software 12 (and/or the cloud 37) as described above. Preferably, the lock box 70 is kept with the patient at all times, for example by attachment to the patient's hospital bed (if applicable). In one example embodiment, the lock box 70 may be configured to require more than one “key” to enable unlocking. For example, the first “key” may be the requirement of a certain patient action confirming association of the lock box 70 with the correct patient (as well as simultaneously confirming patient consent to the procedure). Such patient action may include (but not be limited to) a digitally-captured signature or biometric identification (e.g. iris scan, fingerprint, etc.) transmitted to and recorded by the cloud server 37. In another embodiment, the lock box 70 comprises a portion of the patient's physical chart (e.g. the medical chart may be incorporated into or otherwise attached to the lid 76).

The lock box 70 remains locked until its presence in the treatment room has been verified during the “time out” sequence just prior to treatment. Lock box verification may proceed in a manner substantially similar to the sequence shown and described in relation to FIGS. 6-8 above, but instead of scanning the blade dispenser (or other sterile medical object 14) directly, the user scans the label 84 on the lock box 70. The computer compares the data from the scanned label 84 with the data from the patient ID band 24, verifies the presence of the correct lock box 70 and the specific contents contained therein, and then communicates this verification to the user via a GUI or other display (or communicates a failed verification indicating the presence of a wrong lock box, in which case the procedure would not proceed until the correct lock box is found and verified). After verification of the lock box 70 by the computer (including cloud server 37), the lock box 70 may be opened so that the contents may be retrieved from the internal compartment 74. This may be accomplished in several ways depending upon the technological capabilities of the specific lock box being used. For example, according to one embodiment, the lock box 70 may be manually opened by a user after verification using a physical key that is inserted into a key hole on the lock box 70 (not shown). This option is analogous to the situation previously described by way of example in which the computer indicates to the user that it is safe to proceed (e.g. by causing a visual indicator to appear on the GUI screen), and the user gains access to the safety-blade dispenser by removing the confirmation label. In this situation the computer verifies all relevant information during the time out sequence and communicates to the user that it is safe (or not) to proceed, but cannot prevent the unlocking of the box (or removal of the confirmation label on the safety-blade dispenser).

According to another embodiment, the lock box 70 may be equipped with a numerical keypad (not shown) with which a user may input a code to unlock the box. During the time out procedure and after the correct lock box has been verified, the computer may cause the code for unlocking the lock box 70 to be displayed to the user (e.g. on the display feature of the user's computer 16, handheld device 18, or another display device), who must then manually input the code using the keypad. In this situation, the software 12 acts as a partial gatekeeper in that it will reveal the code only upon verification of the correct lock box, but a user must still manually input the code to unlock the box.

According to another embodiment, the lock box 70 may be equipped with a digital control panel comprising among other things a microprocessor, user interface, and communications module having the ability to communicate with another computer, for example a handheld computer 18 comprising the software 12 and/or cloud server 37 that stores all relevant data pertaining to the procedure. This communication may be using WiFi, Bluetooth, Ethernet, or any other suitable mechanism. Upon verification of the correct lock box 70, the computer may cause the handheld device 18 or cloud server 37 to communicate with the lock box 70, sending the unlock instructions directly to the lock box 70, which the microprocessor executes by unlocking the lock box 70. In this fashion, the software program 12 executed by the computer serves as a full gatekeeper that requires the “time out” to occur. In this instance it may be necessary to ensure that the lock box 70 includes a manual release mechanism in the event that the communication between the handheld device (or cloud server) and the lock box 70 is interrupted.

The lock box 70 may be provided with one or more color indicators to convey certain information that can be recognized and verified on sight, for example such as a color indicator on the lock box 70 that indicates laterality as described above. In addition, a color indicator may be provided that displays a color to match a color-coded patient ID wristband, which may help reduce patient identification errors. Another color indicator may be provided to indicate locked and unlocked status of the lock box. By way of example, the one or more color indicators may be single lights, a string of lights (e.g. LED band), and/or panels. In one embodiment, the entire lock box 70 may be constructed from a material that may be illuminated with one or more colors.

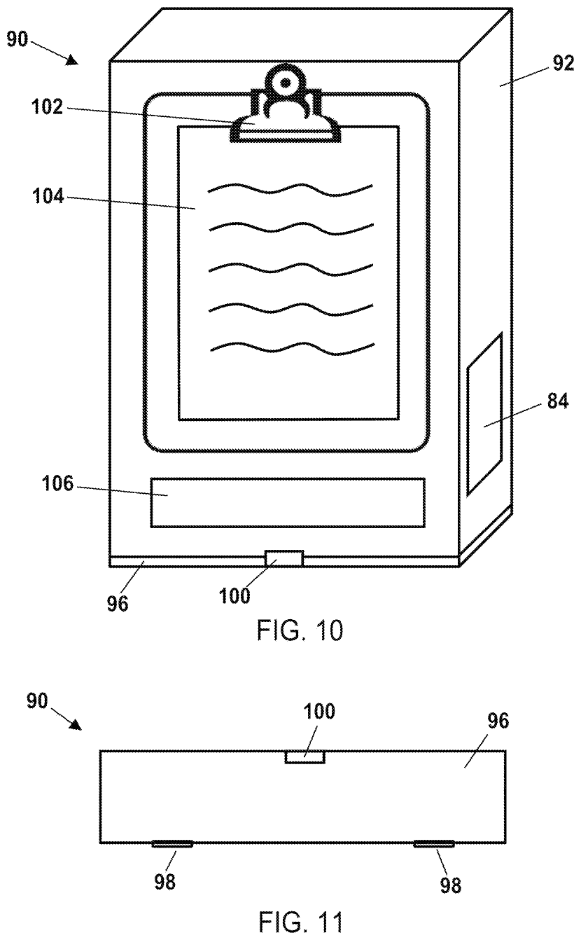

As previously mentioned, the surgical supply carrier or lock box 70 may be constructed such that the lock box forms part of the patient chart and/or clipboard. This combination increases the likelihood that the correct essential medical objects (e.g. surgical blades, implants, etc.) contained within the lock box stay with the patient for the entire duration of the stay in the treatment facility. FIGS. 10-11 illustrate one example of a chart-associated lock box (or “chart box”) 90. By way of example, the chart box 90 shown and described herein may be color coded for laterality as described above, and includes a base 92 having an internal compartment (not shown) and a moveable panel 96 hingedly attached to the base 92 at one end (or side) by one or more hinges 98. The internal compartment is sized and configured to hold one or more medical objects 14 that are specifically selected for a particular patient in a given procedure. The moveable panel 96 is sized and configured to block access to the internal compartment when the moveable panel 96 is in a closed position. The moveable panel 96 is moveable from a closed position in which access to the internal compartment is blocked to an open position in which access to the internal compartment is unencumbered. The chart box 90 further includes a locking mechanism 100 configured to releasably lock the moveable panel 96 to the base 92 when the moveable panel 96 is in the closed position. By way of example, the locking mechanism 100 may comprise a latch on the moveable panel that engages a pin in a recess on the base 92, however any appropriate locking mechanism may be used. Furthermore, although shown and described as a box with a single interior compartment and a lid hingedly attached, the chart box 90 may comprise other types of lockable compartments in addition to or instead of the configuration shown herein, including but not limited to drawers, slideable lids, multiple lockable compartments, and the like. The chart box 90 further includes a spring-biased clip 102 on the exterior surface of the lid for holding paper charts 104, and a confirmation label 84 as described above. Alternatively, the lid may include a computer element (e.g. tablet type computer) including a processor, storage medium, and touch screen display for the purpose of housing a digital patient chart (among other things). In such a case, the software 12 may be installed on the digital clipboard and synced with the software 12 on other computing devices within the medical environment and/or connected to the cloud 37.

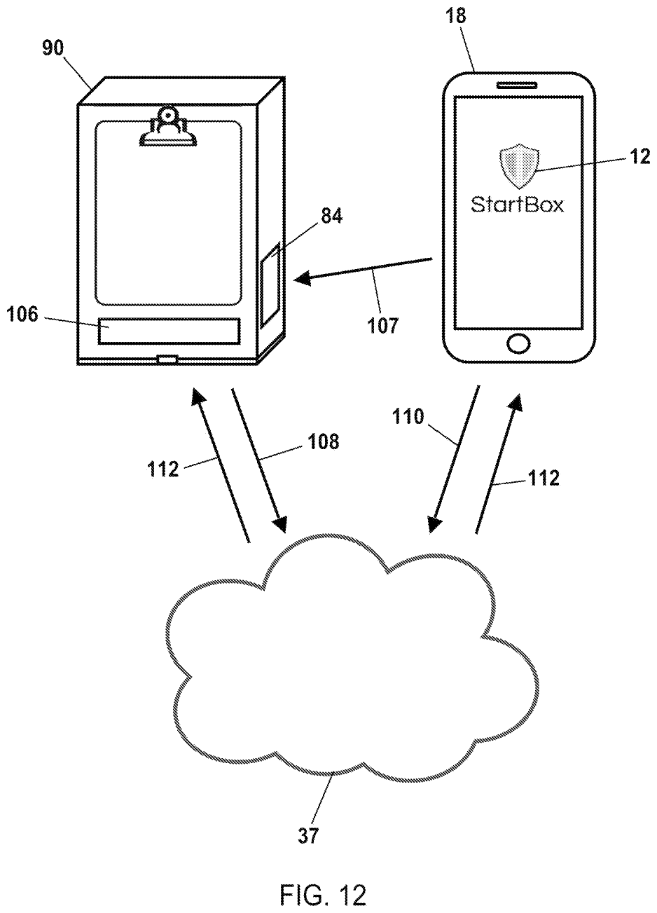

According to one embodiment, multiple “keys” may be needed to unlock the lock box 70 (or chart box 90) after final time out. For example, as described above the first “key” may be established by entering patient/guardian consent directly into the computer via digital signature capture (by way of digital signature bar 106). As illustrated by way of example in FIG. 12, this first key 108 is transmitted to and stored in the cloud server 37. By way of example, the consent may be given before the patient is prepped for surgery. During final timeout sequence (e.g. in the operating room just prior to surgery commencing), the QR code on the lock box 70 (or chart box 90) itself is scanned 107 for verification (as described above using the computer-executed software 12 on a handheld device 18, for example). This second “key” 110 is also transmitted by the computer to the cloud server 37 where the information is verified. If all the information (e.g. patient data, tactical tool data, laterality information, etc.) is positively matched and verified to the patient information stored in the cloud server 37, the cloud server 37 sends an unlock instruction/code 112 to the handheld device 18 that causes the computer to display instructions for the user to act on, or alternatively causes a fully integrated lock box 70/chart box 90 to automatically unlock. The one or more medical objects 14 stored inside the internal compartment may then be accessed and the procedure may begin.

Alternatively, by way of example, the lock box 70 (or chart box 90) may be configured to include an optical scanner in communication with the digital control panel described above. In this example embodiment, the unlock instruction/code 112 may be presented by the computer on the user's device as a digital key in the form of a unique QR code to be scanned by the scanner on the lock box 70, from which the microprocessor in the control panel may receive the unlock instructions and cause the lock box 70 to be unlocked.

By way of example, an additional (or alternative) “key” may involve using technology to enable the computer software program to recognize when the patient has entered the OR for surgery. For example, in addition to the bar code 32 and/or QR code, the patent ID band 24 may include a near-field communication (NFC) chip that has been programmed with or linked to the patent's identification information. The NFC chip may also be associated with the patient's smart-phone, smart-watch, or an independent device secured to the patient and/or patient's bed. A compatible reader within the OR may sense the NFC chip and communicate the ID information contained therein with the computer, which then compares the ID information contained within the NFC chip to the patient's profile stored in the Startbox app. The computer will then cause a positive or negative match indication (e.g. audio, visual, or a combination thereof) to be presented to the medical personnel in the OR. This step would verify that the correct patient has entered the OR in real time.

Although described herein generally in conjunction with a surgical environment, the “time out” feature of the computer software program combined with the confirmation label 26 including a QR code 28 described herein and in the above-referenced '210 PCT (incorporated by reference) may be applicable to a wide variety of medical situations beyond controlling access to sterile objects in a surgical environment, including but not limited to in-patient support, out-patient treatment, dental, and veterinary applications. For example, the lock box 70 may be used in the delivery of medication from a hospital's pharmacy to a patient's room. Pharmacological information can be associated with the QR code on the lock box in the pharmacy. The box is then closed and delivered by appropriate staff (e.g. orderly, nurse, etc.) to the patient's room. Before administering the medication, the staff member may use the scanning feature of the software 12 by way of a handheld computer (as described above) to scan the patient's ID wristband and then scan the label on the lock box 70. Upon verification of a match, the software 12 instructs the computer (either handheld or via cloud server) to send “unlock” instructions to the lock box 70, granting access to the contents inside. In a similar fashion, a lock box 70 as described herein may be used in transportation of patient samples to and from laboratories for analysis. In an ophthalmic procedure, for example, the computer software program may include prescription information for each eye so that the “time out” procedure includes a confirmation of laterality with the correct prescription to ensure the correct ocular implant is used in the correct eye. In dental procedures, the computer software program my include dental implant serial numbers linked with the patient ID band and the lock box, which are confirmed during the final time out procedure.

FIGS. 13-14 are example block diagrams of computer-implemented electronic devices 200, 250 that may be used to implement the systems and methods described in this document, as either a client or as a server or plurality of servers. Computing device 200 is intended to represent various forms of digital computers, such as laptops, desktops, workstations, personal digital assistants, servers, blade servers, mainframes, and other appropriate computers. Computing device 250 is intended to represent various forms of mobile devices, such as personal digital assistants, cellular telephones, smart-phones, and other similar computing devices. In this example, computing device 250 may represent hand-held computing device 18, while computing device 200 may represent stationary computer 16 and/or computing systems that serve as the cloud 37 referenced in this disclosure. The components shown here, their connections and relationships, and their functions, are meant to be examples only, and are not meant to limit implementations described and/or claimed in this document.

Referring to FIG. 13, computing device 200 includes a processor 202, memory 204, a storage device 206, a high-speed interface 208 connecting to memory 204 and high-speed expansion ports 210, and a low speed interface 212 connecting to low speed bus 214 and storage device 206. Each of the components 202, 204, 206, 208, 210, and 212, are interconnected using various busses, and may be mounted on a common motherboard or in other manners as appropriate. The processor 202 can process instructions for execution within the computing device 200, including instructions stored in the memory 204 or on the storage device 206 to display graphical information for a graphic user interface (GUI) on an external input/output device, such as display 216 coupled to high-speed interface 208. In other implementations, multiple processors and/or multiple buses may be used, as appropriate, along with multiple memories and types of memory. Also, multiple computing devices 200 may be connected, with each device providing portions of the necessary operations (e.g., as a server bank, a group of blade servers, or a multi-processor system).

The memory 204 stores information within the computing device 200. By way of example only, the memory 204 may be a volatile memory unit, non-volatile memory unit, or another form of computer-readable medium, such as a magnetic or optical disk (for example).

The storage device 206 is capable of providing mass storage for the computing device 200. In one implementation, the storage device 206 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 204, the storage device 206, or memory on processor 202.

The high-speed interface 208 manages bandwidth-intensive operations for the computing device 200, while the low speed interface 212 manages lower bandwidth-intensive operations. Such allocation of functions is by way of example only. In one implementation, the high-speed interface 208 is coupled to memory 204, display 216 (e.g., through a graphics processor or accelerator), and to high-speed expansion ports 210, which may accept various expansion cards (not shown). In the implementation, low-speed interface 212 is coupled to storage device 206 and low-speed expansion port 214. The low-speed expansion port may include various communication ports (e.g., USB, Bluetooth, Ethernet, wireless Ethernet) and may be coupled to one or more input/output devices, such as a keyboard 218, a printer 220, a scanner 222, or a networking device such as a switch or router 224, e.g., through a network adapter.

The computing device 200 may be implemented in a number of different forms. For example, it may be implemented as a standard server, or multiple times in a group of such servers. It may also be implemented as part of a rack server system. In addition, it may be implemented in a personal computer such as a laptop computer. Alternatively, components from computing device 200 may be combined with other components in a mobile device, such as device 250 (FIG. 14). Each of such devices may contain one or more of computing device 200, 250, and an entire system may be made up of multiple computing devices 200, 250 communicating with each other.

Referring to FIG. 14, computing device 250 includes a processor 252, memory 254, an input/output device such as a display 256, a communication interface 258, and a transceiver 260, among other components. The device 250 may also be provided with a storage device, such as a microdrive or other device, to provide additional storage. Each of the components 250, 252, 254, 256, 258, and 260, are interconnected using various buses, and several of the components may be mounted on a common motherboard or in other manners as appropriate.

The processor 252 can execute instructions within the computing device 250, including instructions stored in the memory 254. The processor may be implemented as a chipset of chips that include separate and multiple analog and digital processors. Additionally, the processor may be implemented using any of a number of architectures. For example, the processor 252 may be a CISC (Complex Instruction Set Computers) processor, a RISC (Reduced Instruction Set Computer) processor, or a MISC (Minimal Instruction Set Computer) processor. The processor may provide, for example, for coordination of the other components of the device 250, such as control of user interfaces, applications run by device 250, and wireless communication by device 250.

The processor 252 may communicate with a user through control interface 262 and display interface 264 coupled to a display 256. The display 256 may be, for example, a TFT (Thin-Film-Transistor Liquid Crystal Display) display or an OLED (Organic Light Emitting Diode) display, or other appropriate display technology. The display interface 264 may comprise appropriate circuitry for driving the display 256 to present graphical and other information to a user. The control interface 262 may receive commands from a user and convert them for submission to the processor 252. In addition, an external interface 266 may be provided in communication with processor 252, so as to enable near area communication of device 250 with other devices. External interface 266 may provide, for example, for wired communication in some implementations, or for wireless communication in other implementations, and multiple interfaces may also be used.

The memory 254 stores information within the computing device 250. The memory 254 can be implemented as one or more of a computer-readable medium or media, a volatile memory unit or units, or a non-volatile memory unit or units. Expansion memory 268 may also be provided and connected to device 250 through expansion interface 270, which may include, for example, a SIMM (Single In Line Memory Module) card interface. Such expansion memory 268 may provide extra storage space for device 250, or may also store applications or other information for device 250. Specifically, expansion memory 268 may include instructions to carry out or supplement the processes described above, and may include secure information also. Thus, for example, expansion memory 268 may be provided as a security module for device 250, and may be programmed with instructions that permit secure use of device 250. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, cause performance of one or more methods, such as those described above. The information carrier is a computer- or machine-readable medium, such as the memory 254, expansion memory 268, or memory on processor 252 that may be received, for example, over transceiver 260 or external interface 266.

Device 250 may communicate wirelessly through communication interface 258, which may include digital signal processing circuitry where necessary. Communication interface 258 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 260. In addition, short-range communication may occur, such as using a Bluetooth, WiFi, or other such transceiver (not shown). In addition, GPS (Global Positioning System) receiver module 272 may provide additional navigation- and location-related wireless data to device 250, which may be used as appropriate by applications running on device 250.

Device 250 may also communicate audibly using audio codec 274, which may receive spoken information from a user and convert it to usable digital information. Audio codec 274 may likewise generate audible sound for a user, such as through a speaker, e.g., in a handset of device 250. Such sound may include sound from voice telephone calls, may include recorded sound (e.g., voice messages, music files, etc.) and may also include sound generated by applications operating on device 250.

The computing device 250 may be implemented in a number of different forms, some of which are shown in the figure. For example, it may be implemented as a cellular telephone. It may also be implemented as part of a smart-phone, personal digital assistant, or other similar mobile device.

Additionally computing device 200 or 250 can include Universal Serial Bus (USB) flash drives. The USB flash drives may store operating systems and other applications. The USB flash drives can include input/output components, such as a wireless transmitter or USB connector that may be inserted into a USB port of another computing device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

1. A system for preventing or tracking a wrong-site error during a surgical procedure associated with an individual interacting within a medical environment, comprising:
a surgical supply carrier comprising at least one internal compartment configured to hold one or more surgical instruments or components to be used during said surgical procedure for said individual, a movable barrier preventing access to said internal compartment when said movable barrier is in a closed position, a locking mechanism configured to maintain the moveable barrier in said closed position until completion of at least two unlocking events, storage media comprising at least one of an electronic profile for said individual and electronic instructions for unlocking said surgical supply carrier, and a communications module configured to enable communication between said storage media and a processor; and
a computer implemented electronic device having a screen for displaying images, an optical scanner for receiving data, a processor operable to execute instructions, and a data storage medium for storing instructions which when executed by the processor cause the processor to:
(1) save and display said electronic profile for said individual;
(2) associate one or more surgical devices with said electronic profile;
(3) receive and store a first data input comprising a first of said at least two unlocking events;
(4) enable an electronically recorded time out to be conducted prior to starting of said surgical procedure associated with said individual, said time out comprising a second of said at least two unlocking events; and
(5) access said storage media of said surgical supply carrier to enable execution of said electronic instructions for unlocking said surgical supply carrier.

2. The system of claim 1, wherein the surgical supply carrier comprises a medical chart of said individual.

3. The system of claim 2, wherein said medical chart is physically attached to said surgical supply carrier.

4. The system of claim 2, wherein said medical chart is electronically stored within said storage media.

5. The system of claim 1, wherein said surgical supply carrier further comprises a display component configured to display data contained within said storage media.

6. The system of claim 1, wherein the optical scanner is configured to retrieve data from at least one of a QR code and a bar code.

7. The system of claim 1, wherein the moveable barrier is hingedly attached to the surgical supply carrier.

8. The system of claim 1, wherein said surgical supply carrier further includes at least one color indicator corresponding to at least one of laterality, patient identification, and lock status.

9. The system of claim 1, wherein execution of said instructions for unlocking said surgical supply carrier causes said moveable barrier to be unlocked electronically.

10. The system of claim 1, wherein execution of said instructions for unlocking said surgical supply carrier causes said computer implemented electronic device to display instructions to a user to manually unlock said moveable barrier.

11. The system of claim 1, wherein said one or more surgical devices to be associated with said electronic profile each include at least one of a bar code and a QR code readable by said optical scanner.

12. The system of claim 1, wherein said first data input comprises digital consent to continue with the surgical procedure, given by one of said individual and a legal guardian of said individual.

13. The system of claim 12, wherein said surgical supply carrier further comprises a digital signature capture element in electronic communication with said storage media, said digital signature capture element configured to capture said digital consent.

14. The system of claim 1, wherein said second of said at least two unlocking events comprises:
using said optical scanner to scan one of a bar code and a QR code affixed to said surgical supply carrier to retrieve data contained within said code;
communicating said retrieved data to said processor; and
comparing said retrieved data to said electronic profile to verify a match between said retrieved data and said electronic profile.

15. The system of claim 1, wherein said electronic profile of said individual comprises at least one of data that identifies said individual, data that identifies said medical procedure scheduled to be performed on said individual, and an indication of procedural laterality.

16. A method for preventing or tracking a wrong-site error during a surgical procedure associated with an individual interacting within a medical environment, comprising:
providing a surgical supply carrier, the surgical supply carrier comprising at least one internal compartment configured to hold one or more surgical instruments or components to be used during said surgical procedure for said individual, a movable barrier preventing access to said internal compartment when said movable barrier is in a closed position, a locking mechanism configured to maintain the moveable barrier in said closed position until completion of at least two unlocking events, storage media comprising at least one of an electronic profile for said individual and electronic instructions for unlocking said surgical supply carrier, and a communications module configured to enable communication between said storage media and a processor;
providing a computer implemented electronic device having a screen for displaying images, an optical scanner for receiving data, a processor operable to execute instructions, and a data storage medium for storing instructions which when executed by the processor cause the processor to save and display said electronic profile for said individual, associate one or more surgical devices with said electronic profile, receive and store a first data input comprising a first of said at least two unlocking events, enable an electronically recorded time out to be conducted prior to starting of said surgical procedure associated with said individual, said time out comprising a second of said at least two unlocking events, and access said storage media of said surgical supply carrier to enable execution of said electronic instructions for unlocking said surgical supply carrier;
associating said surgical supply carrier with said individual;
populating said internal compartment with one or more surgical components;
locking said moveable barrier in a closed position;
capturing said first data input comprising said first of said at least two unlocking events before the individual has been prepped for surgery;
performing said time out including performing said second of said at least two unlocking events while the individual is awaiting commencement of surgery;
retrieving said at least one surgical component from said internal compartment; and
performing said surgical procedure as scheduled on said individual.

17. The method of claim 16, wherein said surgical supply carrier comprises a medical chart of said individual.

18. The method of claim 17, wherein said medical chart is physically attached to said surgical supply carrier.

19. The method of claim 17, wherein said medical chart is electronically stored within said storage media.

20. The method of claim 16, wherein said surgical supply carrier further comprises a display component configured to display data contained within said storage media.

21. The method of claim 16, wherein the optical scanner is configured to retrieve data from at least one of a QR code and a bar code.

22. The method of claim 16, wherein the step of associating said surgical supply carrier with said individual comprises:
affixing an identification label including one of a bar code and a QR code to said surgical supply carrier, said one of a bar code and a QR code including at least one of data that identifies said individual, data that identifies said medical procedure scheduled to be performed on said individual, and an indication of procedural laterality.

23. The method of claim 16, wherein said step of populating said internal compartment with one or more surgical components comprises the sub-steps of:
using said optical scanner to scan one of a bar code and a QR code associated with each surgical component to retrieve identifying data pertaining thereto;
communicating said retrieved identifying data to said processor;
associating said retrieved identifying data with said electronic profile of said individual; and
placing said surgical component into said internal compartment.

24. The method of claim 16, wherein the moveable barrier is hingedly attached to the surgical supply carrier.

25. The method of claim 16, wherein said surgical supply carrier further includes at least one color indicator corresponding to at least one of laterality, patient identification, and lock status.

26. The method of claim 16, wherein execution of said instructions for unlocking said surgical supply carrier causes said moveable barrier to be unlocked electronically.

27. The method of claim 16, wherein execution of said instructions for unlocking said surgical supply carrier causes said computer implemented electronic device to display instructions to a user to manually unlock said moveable barrier.

28. The method of claim 16, wherein capturing said first data input comprises:
obtaining digital consent to continue with the surgical procedure, given by one of said individual and a legal guardian of said individual.

29. The method of claim 28, wherein said surgical supply carrier further comprises a digital signature capture element in electronic communication with said storage media, said digital signature capture element configured to capture said digital consent.

30. The method of claim 16, wherein said performing of said second of said at least two unlocking events comprises:
using said optical scanner to scan one of a bar code and a QR code affixed to said surgical supply carrier to retrieve data contained within said code;
communicating said retrieved data to said processor; and
comparing said retrieved data to said electronic profile to verify a match between said retrieved data and said electronic profile.

31. The method of claim 16, wherein said electronic profile of said individual comprises at least one of data that identifies said individual, data that identifies said medical procedure scheduled to be performed on said individual, and an indication of procedural laterality.
